(19) United States
(12) Patent Application Publication
(10) Pub. No.: US 2005/0154438 A1
(43) Pub. Date: Jul. 14, 2005

(54) Medical electrode system and method

(76) Inventors: Jonathan Andrew Fuller, Inverness (GB); Daniel Strother, Inverness (GB)

Correspondence Address: SUGHRUE MION, PLLC, 2100 PENNSYLVANIA AVENUE, N.W., SUITE 800, WASHINGTON, DC, 20037, US

(21) Appl. No.: 10/498,942

(22) PCT Filed: Dec. 16, 2002
(86) PCT No.: PCT/GB02/05693

(30) Foreign Application Priority Data
Dec. 22, 2001 (GB) 0130906.1

(51) Int. Cl. A61N 1/04

(52) U.S. Cl. 607/148

(57) ABSTRACT

A medical electrode system and method comprises four measuring electrode elements and optional reference electrodes each presenting a skin contactable conducting electrode surface, the four elements adapted to be mounted in use on the skin surface of a selected part of the human or animal body so as to be disposed in a generally orthogonal arrangement with the outputs therefrom being arranged and processed so as to connect the electrode elements either as opposing differential pairs or as potentials with respect to the reference electrode(s) where present

The invention relates to an electrode system and to a method of the use of such an electrode for the measurement of electrical signals from within a human or animal body via the skin surface, in particular for medical such as diagnostic purposes.

It is established in medical practice (which term should be read herein to encompass where appropriate veterinary practice) to apply electrodes to the surface of the human or animal body to obtain information about electrical activity within the body. Such electrode systems are intended to allow monitoring of the weak electrical potentials generated within the body by various physiological processes, in particular for non-invasive diagnostic purposes.

Measurement using surface mounted electrodes can be a particularly effective diagnostic means in relation to the performance of such physiological systems, in particular but not exclusively in relation to electrical signals generated by muscle activity. For example, it is well established that a useful information about the condition and performance of the heart may be obtained by electrocardiography, whereby electrodes placed on the skin surface of the subject in the chest region may be used to collect data relating to the electrical activity of the heart, which data can, when suitably processed, provide a variety of information about heart performance and condition, and a diagnostic tool for a variety of cardiac disorders. Similarly, it is known that by application of electrodes to the abdomen of a pregnant female, electrohysterographic data can be obtained giving an indication of electrical activity within the muscular wall of the uterus, providing information about the condition, function and state of electrical connections within the uterine muscle, and thus providing diagnostic information in relation to labour onset and progress.

In conventional electrode systems, a medical electrode typically comprises a skin contactable conducting portion, typically on a suitable carrier layer adapted to mount the conducting portion in position on the subject's skin, for example by having an adhesive layer. Additional layers, for example for structural purposes or to improve surface contact and conduction (eg conducting gel surface layers) might be included. Typically, a plurality of such measuring electrodes are arrayed across an area of the surface of the human or animal body under test, and optionally one or more additional reference electrodes are similarly applied. Information is obtained and processed from the electrodes.

Such electrode systems have functional limitations. The information processing necessary to obtain meaningful diagnostic data can be complex. Electrical signal amplitudes for many of the medical applications envisaged are very small and subject to significant background noise. Signal analysis for electrodes applied in multiple positions is a practical necessity if information is to be extracted regarding signal direction, speed and like measurements from these very low amplitude electrical signals, such as if necessary for effective diagnosis. Skilled placement of electrodes by a trained practitioner is generally required.

It is an object of the present invention to mitigate some or all the disadvantages of these prior art systems.

It is a particular object of the present invention to provide an improved medical electrode, and an improved method of use of such an electrode, which enhances diagnostic potential by exhibiting an enhanced sensitivity to electrical potential generated within the human or animal body.

It is a particular object of the present invention to provide an electrode system, method and analysis regime enabling extraction of amplitude, direction and speed measurements from the weak electrical potentials generated within the body for subsequent diagnostic use.

Thus, in accordance with the present invention in its first aspect, an electrode system in particular for medical purposes comprises four measuring electrode elements each presenting a skin contactable conducting electrode surface, the four elements adapted to be mounted in use on the skin surface of a selected part of the human or animal body so as to be disposed in a generally orthogonal arrangement. The electrode system may include one or more additional electrode elements presenting skin contactable electrode surfaces intended to serve as reference electrodes. In particular, a fifth electrode is provided as a reference electrode generally centrally within the orthogonal array of the four measurement electrode elements or otherwise on a midline between two pairs of adjacent measuring electrodes.

The electrode system further includes output means to enable an output signal to be extracted from each of the measurement electrodes and reference electrodes where applicable. Preferably, the output means from the four measurement electrode elements are arranged as to connect the electrode elements either as opposing differential pairs or as potentials with respect to the reference electrode(s) where present.

An electrode arrangement in accordance with the present invention offers significant advantages over previous electrode systems, offering significantly enhanced ability to extract amplitude, direction and speed measurements from the weak electrical signals produced within the body of the subject. This is obtained by suitable analysis exploiting features inherent to the orthogonal arrangement, so that the complex individual electrode elements suggested in some prior art systems are not necessary. In particular, each electrode element need not be a complex combination electrode, but is preferably a single electrode presenting a single contact surface.

Electrode elements making up the system may be of any suitable design which allows direct contact to the skin of the subject with low resistance levels. Suitable prior art electrodes, for example of a wet gel or reusable type, will readily suggest themselves as being appropriate for modification into the arrangement of the invention.

In particular, each electrode element preferably comprises a skin contactable conducting electrode portion mounted upon a carrier layer, which carrier layer is preferably adapted to effect engagement, in particular temporary releasable engagement to allow for reuse, of the electrode onto the skin surface of the subject, for example by having a skin contactable adhesive coating on a lower surface thereof.

The carrier portion may comprise a single layer of material, or may comprise multiple layers conferring other desirable properties. For example, the carrier portion may comprise at least a skin contactable carrier layer and at least one backing layer. The skin contactable carrier layer is conveniently an electrical insulator, and conveniently comprises a skin-adhesive layer of flexible polymeric material. The carrier layer is conveniently provided with an aperture through which the conductive electrode layer may make electrical contact with the subject's skin.

Each electrode conducting portion is made of a suitable conducting material, for example a metallic conducting material, adapted to make direct or indirect low resistance contact with the subject's skin. It is likely in a preferred embodiment that such an electrode will be deposited by screen printing on any suitable non-conducting flexible substrate. As will be known from the prior art, an indirect contact through a conducting gel to lower the resistance between skin and conductor is frequently preferred.

For example, a wet conducting gel is first applied to the subject's skin before the electrode conducting portion is applied thereto. Alternatively, the electrode itself incorporates a conducting gel layer on the lower surface of the carrier portion to effect a low resistance contact between the electrode conducting portion and the skin. For example, the carrier portion comprises an insulating skin contactable carrier layer apertured to expose the electrode conducting portion as above described, with the said conducting gel layer being disposed on the lower surface of the electrode generally coextensive with the said aperture. The conducting gel layer may be integral with the carrier layer, or may be provided with its own support layer, again for example an insulator similarly apertured, to be mounted upon the carrier layer to effect the necessary connection

Each electrode element may be provided with a separate carrier portion, but preferably the electrode system incorporates structural means to facilitate or ensure correct orthogonal disposition of the four measurement electrode elements. Conveniently this is achieved in that the conducting portions making up the four measurement electrode elements are disposed in a generally orthogonal arrangement in fixed association with a single, common or integral carrier member. For example the conducting portions making up the four measurement electrode elements may be mounted on a common carrier layer as above described, or separate carrier layers might be mounted on a common backing layer, or a single carrier layer might be provided comprising the primary electrode contacts in suitable array, with conducting skin contact pads being attachable thereto, for example comprising conducting portions mounted on insulating support portions as above described.

Reference electrodes, where present, may be provided in similar integral carrier mountings, or may be provided for separate mounting as desired. In particularly preferred embodiment, a fifth, reference electrode is disposed on a common carrier member with the four orthogonally disposed measuring electrodes, in particularly generally at the centre thereof or otherwise on a midline between two pairs of adjacent measuring electrodes.

The spacing of the electrodes is dependent on the biological parameter to be measured, but for most applications will generally be not less than 25 mm and not greater than 75 mm.

In accordance with the further aspect of the invention, a method of measuring electrical activity from within the human or animal body comprises the use of the electrode system hereinbefore described. The method in particular involves the use by attachment of the electrode system as hereinbefore described to the skin surface, and the measurement of electrical potentials obtained thereby.

In particular, the method comprises placing four measuring electrode elements into low resistance skin contact with the skin surface of a human or animal body in the region to be tested, such that the four electrode elements are disposed in a generally orthogonal arrangement; optionally placing additional reference electrode elements into similar skin contact, and in particular placing a fifth reference electrode element into contact with the skin generally at the centre of the array of four measuring electrode elements or otherwise on a midline between two pairs of adjacent measuring electrodes; retrieving electrical signals from the four measuring electrode elements; analysing electrical signals from the four electrode elements either as opposing differential pairs or as potentials with respect to the reference electrode elements.

The method is particularly suited to obtaining improved diagnostic information from the relatively weak electrical potentials generated at the skin surface for example representative of cardiac or uterine activity.

In one preferred embodiment of the method, a method of monitoring uterine activity, and in particular a method of diagnosing labour or predicting the onset of labour, comprises use of the above method to attach electrodes to the abdominal wall in the vicinity of the uterus for a sufficient period to record electrical activity, acquiring data corresponding to the electrical activity, analysing the data to produce an analysis of uterine activity with reference to pre-recorded reference data and/or pre-determined reference parameters to obtain information about uterine electrical activity.

The method thus applies the electrodes of the present invention to the diagnostic method described in WO 01/45555.

In particular the method comprises the step of analysing the data by a power frequency analysis technique for example by performing a spectral analysis of power density of electromyographic potentials. Preferably analysis comprises producing a power spectrum, and performing a load average ratio analysis of at least one low and one high frequency range.

Preferably, the data analysis is adaptive over time for a given patient, and for example comprises making a comparison of changes in uterine electrical activity occurring progressively through pregnancy to produce diagnostic information. In particular the method comprises making a comparison of changes in uterine electrical activity occurring progressively through the pre-labour phase of parturition against suitable reference parameters to determine an indication of imminence of preparedness for labour, and thus to serve as a predictive tool for prediction of the onset of labour, and especially premature labour before contractile symptoms are evident.

In a further aspect of this embodiment, a device for monitoring uterine state in a human or non-human mammal comprises a basic electrode system as hereinbefore described for attachment to the abdominal wall in the vicinity of the uterus, a means for data acquisition and a data analysing means for analysing the acquired data in accordance with the above analysis method, and may also comprise a display adapted to display this analysed data to a user for example as a diagnostically useful result.

In another preferred embodiment of the method a method of monitoring cardiac activity comprises use of the above method to attach electrodes to the chest wall for a sufficient period to record electrical activity, acquiring data corresponding to the electrical activity, analysing the data to produce an analysis of cardiac activity, for example with reference to pre-recorded reference data and/or pre-determined reference parameters to obtain information about cardiac function.

The method thus applies the electrodes of the present invention to the diagnostic method described in GB 0130906.1.

In particular the method therefore comprises the step of analysing the data by way at least of the steps of producing a frequency and/or signal intensity based data characterisation and inferring and outputting therefrom a result representative of the heart beat rate of the subject. For example the result is derived by identifying a peak in the frequency and/or signal intensity based data characterisation. For example the data is analysed to produce a power density spectrum, identify the peak power frequency therefrom within a predetermined range corresponding to a range of possible heart beat rates, derive thereby a result representative of the heart beat rate of the subject.

Preferably this analysis includes the steps of de-noising, signal isolation or conditioning the digitised data and/or performing a fast Fourier transform, wavelet transform or other mathematical transform on the acquired data to produce the said frequency and/or signal intensity based data characterisation (such as a power density spectrum) and derive a heart rate.

The method bears similarities to conventional electrocardiographic techniques, but exploits the advantages of the new electrode architecture of the invention. The method is not designed to produce a full or partial ECG analysis/recognition but instead to infer the heart beat rate from a simpler analysis and relies on the surprising realisation that an effective indication of simple heart beat rate can be obtained without the need to resolve a full signal in the manner conventionally followed by ECG techniques, especially when the collection sensitivity of the present electrode system is employed.

In particular, in the prior art where conventional ECG techniques are used, it is necessary to resolve fully a QRS signal and to measure the beat rate by an analysis of the R waves therein. To get an effective measurement, a huge amount of extraneous data is processed, and the apparatus tends to be complex, large, and require operation by a skilled practitioner. By contrast, in this embodiment of the method, the electrical activity of the heart beat is not resolved and measured directly, but rather a representative “proxy” measure is obtained. The raw electrical activity data is acquired from the electrodes, and appropriate mathematical techniques are used to produce a frequency and/or signal intensity based data characterisation such as a power density spectrum. The heart rate is then inferred by interrogation of these data. This does not require a resolution of the detailed electrical cardiac activity to pinpoint R waves and to use these to measure heart rate. The resulting method, and any apparatus used to put it into practice, can be greatly simplified, potentially made much more compact for home use, and potentially be available for non-expert application.

In a further aspect of this embodiment, a device for monitoring cardiac state and in particular heart rate in a human or animal comprises a basic electrode system as hereinbefore described for attachment to the chest wall, a means for data acquisition and a data analysing means for analysing the acquired data in accordance with the above analysis method, and may also comprise a display adapted to display this analysed data to a user for example as a diagnostically useful result, for example as an inferred heart rate.

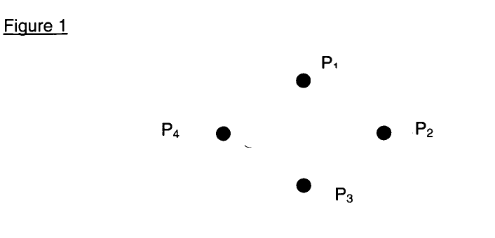

The invention will now be described by way of example and with reference to FIGS. 1 to 3 of the accompanying drawings in which:

FIG. 1 is a schematic illustration of an array of four measurement electrodes in accordance with the invention set up as a bridge;

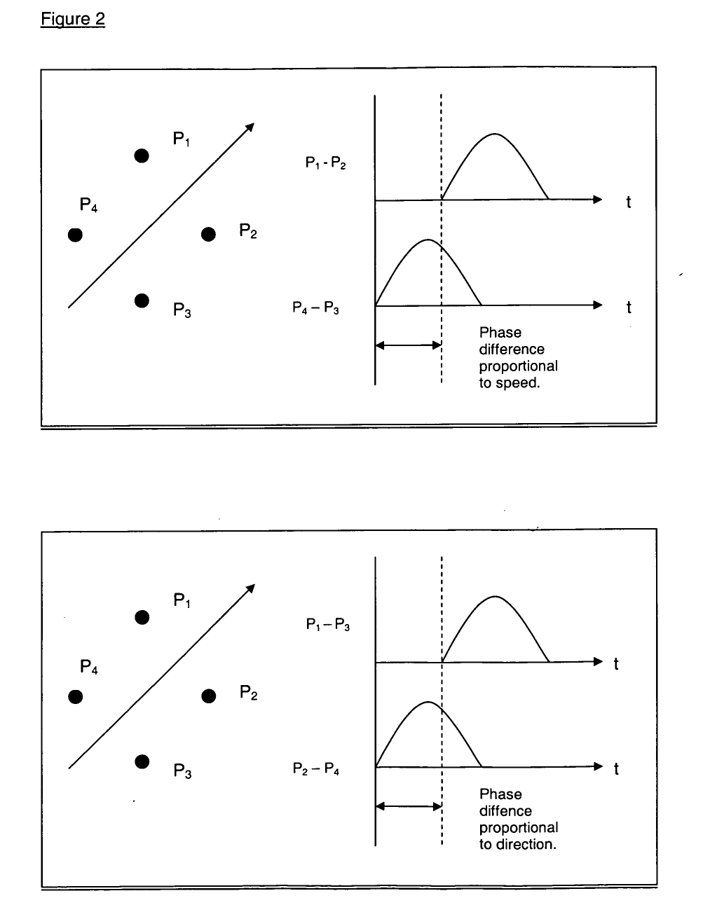

FIG. 2 is an illustration of the use of adjacent pair measurements to obtain time based phase different information;

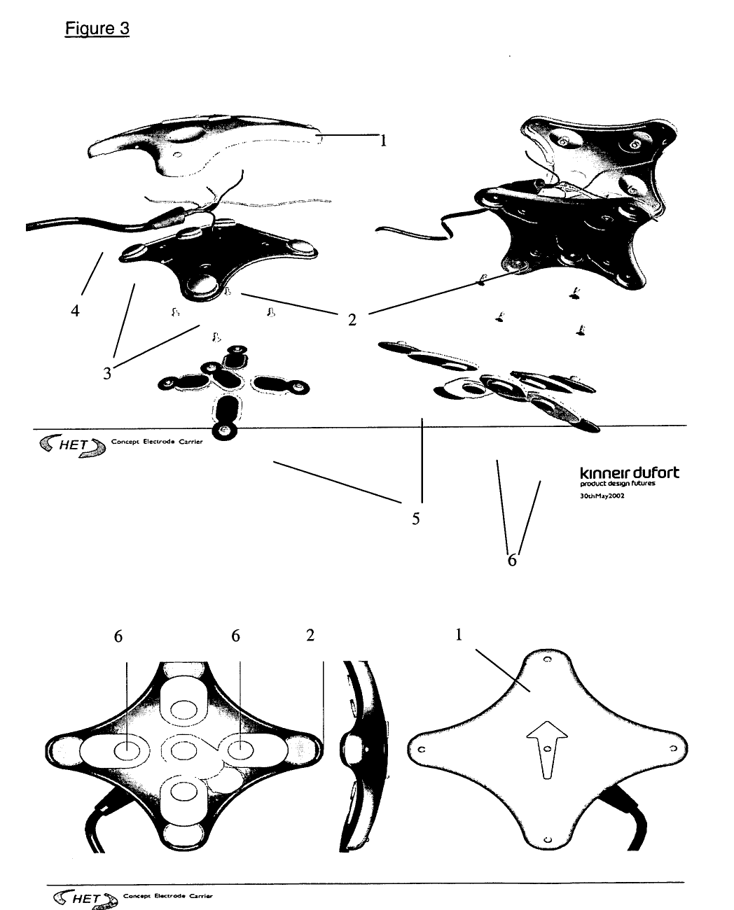

FIG. 3 illustrates a suitable electrode assembly in accordance with the invention.

Although of the invention should not be considered limited to a particular data analysis method, the general discussion below of a possible data analysis procedure with reference to FIGS. 1 and 2 exploits the potential of the invention and is illustrative of its advantages.

Recording of the data can be either continuous (time based) or discrete. The signals can then be treated by the following three analysis methods.

First as a bridge. Each of the potentials is obtained as shown in the diagram of FIG. 1.

The output is then:
((P₁-P₄)−(P₁-P₂))−((P₂-P₃)−(P₄-P₃))

Which is the equivalent output potential of the bridge.

Second, as a set of potential differences. The output in this case is the differences between adjacent pairs which can then be analysed independently.

Third, as a time based phase difference. The output in this case is based on the differences between adjacent pairs as before, but the signals are referenced to time. As an electrical wave passes through the electrode bridge, the relating potentials will be different at different times. Hence, by analysing this the phase difference can be determined allowing the absolute speed and direction of the potential wave to be calculated. This is illustrated in FIG. 2.

The device using these or other suitable analysis offers the following advantages over prior art electrode systems not so arranged:
1) The sensitivity is higher than current single/paired electrodes as the electrodes are used as a bridge. By using them differentially in this way, sensitivity is enhanced by looking at the potential balance, not absolutes.
2) Low noise—electrodes are always used in differential pairs so electrical noise is reduced. This combined with 1) above, means higher signal to noise ratio improving measuring accuracy. This is important when trying to detect extremely low level signals.
3) Information is enhanced by the ability to use the pairs to determine phase giving rise to analysis of speed and direction of the potential wave. This gives enhanced diagnostic ability.

FIG. 3 illustrates an example electrode assembly in accordance with the invention, in exploded view (upper) and assembled (lower).

The electrode assembly comprises a polymeric carrier layer (2) which is contoured to sit against the skin of a subject and is preferably adapted to effect engagement onto the skin surface of the subject by having a skin contactable adhesive coating on a lower surface. Incorporated into the layer (2) are five electrode contacts (3) comprising four measuring electrodes in a square array at the corners of the carrier layer (2) and a fifth reference electrode lying within the square array. In the embodiment, the spacing between adjacent electrodes is 50 mm, the longest dimension of the complete assembly being 122 mm.

The electrodes are connected via connection cable (4) to enable monitoring of electrical signals generated within the subject's body. Connection is in accordance with the principles set out above, for example with the four measuring electrodes arranged in a bridge pair array. This enhances the sensitivity of signal monitoring for the reasons described above.

In the embodiment shown electrical connection between the electrode contacts (3) and the subject is via contact pads (5) which are engaged upon the lower face of the carrier layer (2). The contact pads (5) comprise skin contactable conducting portions (6) in electrical contact with the electrode contacts (3) and support portions of suitable non-conducting material.

For example the conducting portion (6) may be a conducting gel layer disposed so as to lie on the lower surface of the assembly to effect a low resistance contact between the electrode contact (3) and the skin of the subject. The support portion may be an insulating polymer apertured below the electrode contact, with the conducting gel layer being disposed on the lower surface of the electrode assembly generally coextensive with the aperture to effect the necessary electrical connection. Alternatively, electrodes may be of other design and a wet conducting gel may be first applied to the subject's skin before the assembly is applied thereto.

The assembly is completed by addition of a relatively rigid cover plate (1).

The assembly is simple to attach to the subject's body surface, ensures that electrodes are correctly arrayed, and is therefore easy to use to obtain enhanced readings of electrical activity from within the subject's body.

1. An electrode system in particular for medical purposes comprises four measuring electrode elements each presenting a skin contactable conducting electrode surface, the four elements adapted to be mounted in use on the skin surface of a selected part of the human or animal body so as to be disposed in a generally orthogonal arrangement.

2. An electrode system in accordance with claim 1 further including one or more additional electrode elements presenting skin contactable electrode surfaces intended to serve as reference electrodes.

3. An electrode system in accordance with claim 2 wherein a fifth electrode element is provided as a reference electrode generally centrally within the orthogonal array of the four measurement electrode elements.

4. An electrode system in accordance with claim 1 further including output means to enable an output signal to be extracted from each of the electrodes, the output means from the four measurement electrode elements being arranged as to connect the electrode elements either as opposing differential pairs or as potentials with respect to the reference electrode(s) where present.

5. An electrode system in accordance with claim 1 wherein each electrode element comprises a skin contactable conducting electrode portion mounted upon a carrier layer, which carrier layer is adapted to effect engagement of the electrode onto the skin surface of the subject.

6. An electrode system in accordance with claim 5 wherein each electrode element comprises a skin contactable adhesive coating on a lower surface thereof.

7. An electrode system in accordance with claim 5 wherein the carrier portion comprises at least a skin contactable carrier layer and at least one backing layer.

8. An electrode system in accordance with claims 5 wherein the skin contactable carrier layer is an electrical insulator, and comprises a skin-adhesive layer of flexible polymeric material provided with an aperture through which the conductive electrode layer may make electrical contact with the subject's skin.

9. An electrode system in accordance with claims 5 wherein each electrode itself incorporates a conducting gel layer on the lower surface of the carrier portion to effect a low resistance contact between the electrode conducting portion and the skin.

10. An electrode system in accordance with claim 1 incorporating structural means to facilitate or ensure correct orthogonal disposition of the four measurement electrode elements.

11. An electrode system in accordance with claim 10 wherein the conducting portions making up the four measurement electrode elements are disposed in a generally orthogonal arrangement in fixed association with a single, common or integral carrier member.

12. An electrode system in accordance with claim 11 wherein a fifth, reference electrode is disposed on a common carrier member with the four orthogonally disposed measuring electrodes, generally at the centre thereof.

13. An electrode system in accordance with claim 1 wherein the spacing between measuring electrodes is not less than 25 mm and not greater than 75 mm.

14. A method of measuring electrical activity from within the human or animal body comprises the use of the electrode system in accordance with claim 1 by attachment of the electrode system to the skin surface of a human or animal body, and the measurement of electrical potentials obtained thereby.

15. A method of measuring electrical activity from within the human or animal body comprises placing four measuring electrode elements into low resistance skin contact with the skin surface of a human or animal body in the region to be tested, such that the four electrode elements are disposed in a generally orthogonal arrangement; retrieving electrical signals from the four measuring electrode elements; analysing electrical signals from the four electrode elements either as opposing differential pairs or as potentials with respect to the reference electrode elements.

16. The method of claim 15 further comprising placing an additional reference electrode element into similar skin contact generally at the centre of the array of four measuring electrode elements.

17. The method of claims 14 used as a method of monitoring uterine activity, and in particular a method of diagnosing labour or predicting the onset of labour, comprising attachment of the electrodes to the abdominal wall in the vicinity of the uterus for a sufficient period to record electrical activity, acquiring data corresponding to the electrical activity, analysing the data to produce an analysis of uterine activity with reference to pre-recorded reference data and/or pre-determined reference parameters to obtain information about uterine electrical activity.

18. The method of claim 17 comprising the steps of analysing the data by a power frequency analysis technique for example by performing a spectral analysis of power density of electromyographic potentials. Preferably analysis comprises producing a power spectrum, and performing a load average ratio analysis of at least one low and one high frequency range.

19. The method of claim 18 wherein the data analysis is adaptive over time for a given patient, and comprises making a comparison of changes in uterine electrical activity occurring progressively through pregnancy to produce diagnostic information.

20. The method of claims 14 used as a method of monitoring cardiac activity comprises attachment of electrodes to the chest wall for a sufficient period to record electrical activity, acquiring data corresponding to the electrical activity, analysing the data to produce an analysis of cardiac activity, for example with reference to pre-recorded reference data and/or pre-determined reference parameters to obtain information about cardiac function.

21. The method of claim 20 comprising the step of analysing the data by way at least of the steps of producing a frequency and/or signal intensity based data characterisation and, inferring and outputting therefrom a result representative of the heart beat rate of the subject.

22. The method of claim 21 comprising the steps of analysing the data to produce a power density spectrum, identify the peak power frequency therefrom within a predetermined range corresponding to a range of possible heart beat rates, derive thereby a result representative of the heart beat rate of the subject.

23. The method of claim 22 comprising the steps of de-noising, signal isolation or conditioning the digitised data and/or performing a fast Fourier transform, wavelet transform or other mathematical transform on the acquired data to produce the said frequency and/or signal intensity based data characterisation (such as a power density spectrum) and derive a heart rate.
